(19) United States
(12) Patent Application Publication
(10) Pub. No.: US 2020/0143938 A1
(43) Pub. Date: May 7, 2020

(54) SYSTEMS AND METHODS FOR SCHEDULING ON-DEMAND MEDICAL APPOINTMENTS

(71) Applicants: Alireza Haghighi Kakhki, Brookline, MA (US); Mohammad Farivar, Chestnut Hill, MA (US); Reza Mollaaghababa, Natick, MA (US)

(72) Inventors: Alireza Haghighi Kakhki, Brookline, MA (US); Mohammad Farivar, Chestnut Hill, MA (US); Reza Mollaaghababa, Natick, MA (US)

(21) Appl. No.: 16/673,327

(22) Filed: Nov. 4, 2019

Related U.S. Application Data
(60) Provisional application No. 62/755,851, filed on Nov. 5, 2018.

(51) Int. Cl. G16H 40/20; G06Q 10/10

(52) U.S. Cl. CPC G16H 40/20; G06Q 10/1095

(57) ABSTRACT

Methods and systems to facilitate scheduling on-demand medical appointments with medical professionals are disclosed. In one embodiment, a computer-implemented system can include a scheduling server configured to communicate with a plurality of patient devices and a plurality of physician devices. The scheduling server program can include a first module for receiving requests from said patient devices for information regarding at least one available physician having at least one available appointment time slot, a second module for receiving available patient appointment time slots from said plurality of physician devices, a third module for searching in response to a request from at least one of said patient devices to identify one or more available appointment time slots, and a fourth module for receiving information regarding said available time slots and for transmitting said information to said at least one requesting patient device.

### CROSS-REFERENCE TO RELATED APPLICATIONS

This application claims the benefit of U.S. Provisional Application 62/755,851, filed Nov. 5, 2018. The entire contents of this application are incorporated by reference herein.

### FIELD

The present invention relates generally to methods and systems that facilitate the scheduling of medical appointments, and more particularly to methods and systems that can be used by patients to make on-demand medical appointments with a variety of medical professionals, such as physicians and dentists.

### BACKGROUND

Each year, patients schedule millions of appointments with medical professionals. Such appointments are made typically well in advance, e.g., a few months in advance. In many cases, a patient may be not be able to attend a previously scheduled appointment, e.g., due to a family emergency or otherwise. Although the patient may notify the medical professional of her inability to keep the appointment, such notification may be provided only a short time, e.g., 24 hours or less, prior to the scheduled appointment. Such short notices can hamper the ability of the medical professional to schedule appointments for other patients in the time slots that have become available due to missed appointments. Such missed appointments can result in loss of revenue. In fact, some data suggests that missed medical appointments cost the U.S. healthcare system about 150 billion dollars each year.

Further, a patient may need an appointment with a medical professional, for example, a family physician or a specialist, over a time period that is significantly less than the typical time periods during which the patient's own physician has available appointments. For example, a patient may desire to see a medical professional over the next 24 or 48 hours. Although many qualified medical professionals may have available time slots in which they can accommodate such a patient, the present systems do not allow facile connection of the patient with such medical professionals.

Accordingly, there is a need for methods and systems that can allow making medical appointments on-demand.

### SUMMARY

In one aspect, a system for scheduling medical appointments is disclosed, which comprises a scheduling server, implemented on a digital processor, that is configured to communicate with a plurality of patient devices associated with a plurality of patients and a plurality of physician devices associated with a plurality of physicians. The scheduling server can include a first module for receiving requests from the patient devices for information regarding at least one available physician having at least one available appointment time slot, a second module for receiving available patient appointment time slots from said plurality of physician devices, said second module storing information regarding said available appointment time slots, a third module in communication with said first module for searching said database in response to a request from at least one of said patient devices to identify one or more physicians and one or more available appointment time slots associated with said one or more physicians that satisfy said request, and a fourth module in communication with said third module for receiving information regarding said identified physicians and the respective available time slots from said third module and for transmitting said information to said at least one requesting patient device.

In some embodiments of the above system, each of the physician devices can include an appointment information module for communicating available appointment time slots to said scheduling server, and an appointment scheduling module for scheduling an appointment at a time slot selected by a patient.

Further, in some embodiments, each patient device can include a first module for communicating a patient's request for identifying at least one physician having at least one available appointment time slot within a desired temporal period to said scheduling server, said first module further configured for receiving information regarding available physicians and associated available appointment time slots from said scheduling server, and a graphical user interface (GUI) in communication with said first module for presenting said information regarding available physicians and associated available time slots to the patient. In some embodiments, each of the patient devices can further include a second module in communication with said GUI to receive a patient's selection of an available physician and a respective available time slot and to communicate said selection to said scheduling server.

In some embodiments, the GUI employs a graphical element for presenting the information regarding available physicians to the patient. In some embodiments, the graphical element presents a boundary line circumscribing an area within a maximum distance from an address provided by the user of the system, such as a patient (e.g., patient's residential address). In some such embodiments, the graphical element can further present one or more icons within the area circumscribed by the boundary indicative of one or more physicians that have available appointment time slots. In some such embodiments, each icon can be presented at a distance from the address (e.g., the patient's address) that is proportional to an actual distance of the address of the presented physician from the patient's address.

In some embodiments, each icon is configured such that its selection can result in the presentation of information regarding the physician associated with that icon to the patient. In some embodiments, the GUI can superimpose a map on the circumscribed area and mark the patient's address and one or more addresses of said one or more physicians on that map.

In a related aspect, a computer-implemented method of scheduling medical appointments is disclosed, which comprises using at least one digital data processor to perform the following steps: receiving at least one request from at least one patient device for information regarding at least one available physician having at least one available appointment time slot, receiving from one or more physician devices information regarding one or more available appointment time slots associated with said one or more physicians, comparing said available appointment time slots with said patient-requested time slot to determine if any of the available appointment time slots satisfies the patient's request, and presenting at least one available appointment time slot satisfying the patient's request, if any, to the patient.

In some embodiments of the above method, the received one or more available time slots correspond to appointment time slots made available via patient cancellation.

In a related aspect, a computer-implemented system for scheduling medical appointments, which comprise a scheduling system comprising a digital data processor for receiving at least one request from a patient for a medical appointment with a medical professional at a selected date and time interval, an information-receiving system comprising a digital data processor and configured for receiving information regarding one or more physicians having available appointment time slots at said selected date and time interval and communicating said information to the scheduling system, where said scheduling system presents said available appointment time slots to said patient.

Further understanding of various aspects of the present teachings can be obtained by reference to the following detailed description in conjunction with the associated drawings, which are described briefly below.

### BRIEF DESCRIPTION OF THE DRAWINGS

FIG. 1 schematically depicts a system according to an embodiment for scheduling on-demand appointments with medical professionals,

FIG. 2 schematically depicts a scheduling server according to an embodiment of the present teachings,

FIG. 3A schematically depicts a patient client application executing on a patient device in accordance with an embodiment of the present teachings,

FIG. 3B schematically depicts an example icon associated with a patient client application, which can be presented to a patient on a patient device,

FIG. 3C illustrates a graphical element, which a patient can employ to provide information to a patient client application regarding a desired medical appointment,

FIG. 3D illustrates a pull-down menu presented to a patient by a patient client application, which the patient can employ to select a medical specialty needed by the patient,

FIG. 3E illustrates a graphical element in the form of a calendar, which a patient can employ to indicate the date and time of a desired medical appointment,

FIG. 4 schematically depicts an example of an implementation of a client physician application,

FIG. 5 schematically depicts a graphical user interface presented by a patient client application to a patient upon launching of the patient client application,

FIG. 6 depicts an example of a graphical element for soliciting information from a patient for constructing a patient profile,

FIG. 7 depicts an example of a graphical element for soliciting information from a medical professional (e.g., a physician) for constructing a profile of the medical professional,

FIG. 8A depicts an icon associated with an appointment module of a physician client application,

FIG. 8B depicts a graphical element in the form of a calendar that can be employed, e.g., by a medical professional or her assistant, to identify available appointment time slots (including those associated with canceled appointments),

FIG. 9 depicts a graphical element for presenting search results in response to a patient's request for a medical appointment to the patient,

FIG. 10 depicts a another graphical element for presenting search results in response to a patient's request for a medical appointment to the patient, and

FIG. 11 schematically depicts an example of an implementation of any of the scheduling sever as well as patient and physician client applications.

### DETAILED DESCRIPTION

The present invention discloses generally methods and systems for scheduling medical appointments on-demand. As discussed in more detail below, a patient device can include a patient client application (herein also referred to as an “patient app”) that allows the patient to communicate with a scheduling server, which stores up-to-date information regarding available medical appointments, to request scheduling a medical appointment. By way of example, in some embodiments, to request an appointment, the patient can launch the patient app, which presents a map to the patient indicating the patient's location and the location of nearby physicians (or other medical professionals) who have available appointments within a defined time period (e.g., a time period extending from the time the patient launches the app to the next 24 hours). In some cases, the patient client app presents all nearby physicians and in other cases nearby physicians satisfying certain criteria, e.g., physicians having a selected specialty, e.g., all nearby dermatologists, are presented, e.g., based on the patient's preferences. In some embodiments, the nearby physicians are those who are located at a distance less than a predefined maximum distance from the patient's location. Such a maximum distance can be set by the patient, or can be a default maximum distance employed by the patient app.

As discussed in more detail below, in some aspects, a system according to the present invention provides an improved scheduling system having a subsystem that allows leveraging patient cancellations associated with a medical practice to provide a medical appointment marketplace that allows substantially real-time scheduling of medical appointments. In this respect, the present invention is an improvement to the technical aspects of a processor-based system for scheduling medical appointments.

The term “patient” is used herein broadly to refer to a user of a system according to the present teachings. In many instances, such a user can be a patient seeking medical advice. In other cases, the user may not be a patient. For example, a user of the system can be an individual who is searching for medical professional on behalf of a patient.

In some embodiments, in order to use the system according to the present teachings, a patient may be required to have a valid health insurance or agree to pay all the incurred costs. In some embodiments, a patient can sign up with the patient client application by providing certain information, such as, social securing number (SSN), health insurance and credit card information, as well as providing consent for utilizing the system. In some embodiments, the patient can also enter preliminary medical/health information into the patient client application. In some embodiments, a scheduling server in communication with the patient client application can assign a unique identification to the patient. Further, in some such embodiments, the scheduling server can communicate with an electronic health system (EHR), with the patient's consent, to download health information regarding the patient, such as clinical reports, biochemical and imaging tests, etc. The scheduling server can then be utilized by the patient or a physician (or other medical professional) with whom the patient has an appointment to access such health data.

Further, in some embodiments, a physician can utilize a system according to the present teachings to receive requests for medical appointments only if the physician has a valid medical license for practicing medicine in the state in which the physician practices as well as a valid malpractice insurance. In some embodiments, a physician seeking to participate in an on-demand appointment service according to the present teachings will be required to submit such information, and optionally other information (e.g., whether the physician has been disciplined by a state medical board). If the physician passes a minimum set of requirements, the physician can be assigned a unique identification to participate in the on-demand appointment service according to the present teachings. In some embodiments, a system according to the present teachings allows a physician to indicate physician's preferences for scheduling appointments. For example, a physician may indicate that she/he is or is not available for visiting patients at their homes. In addition or alternatively, the physician can provide information regarding her/his medical specialty, number of years in practice, insurance plans that the physician accepts, languages spoken, the address of the medical facility at which the physician practices, etc. As discussed in more detail below, the scheduling server can employ such physician information to build a profile of the physician.

As noted above, in response to a patient's request, the patient can be presented with one or more physicians (or other medical professionals). The patient can select one of the presented physicians (or other medical professionals), e.g., by clicking on an icon representing that physician. In some embodiments, in response to the selection of a physician, the patient client app will present the following information regarding the selected physician to the patient: name, gender, specialty, years in practice, rating, the fee, acceptable insurance plans, and the office address (for in-office physicians), and languages spoken.

In some embodiments, a patient can sort the list of physicians based on the above-mentioned information. For example, the patient client app can allow the patient to sort the presented physicians based on the years in practice.

In some embodiments, the patient can select the date/time for an appointment, and the patient client app can present those physicians having available appointment during the selected date/time to the patient.

The patient can then use the client patient app to request an appointment (e.g., a visiting or an in-office) appointment with one of the physicians (or other medical professionals) having an available appointment. In some embodiments, the patient can request an appointment with a specific physician (or other medical professional) or simply make a request for a physician (or other medical professional) having an available appointment time slot and allow the client patient app to choose a physician (e.g., based on the patient's stated preferences, such as years in practice, proximity to the patient's location, etc.). The client physician app can send the appointment request to the scheduling server, which will in turn communicate with a device associated with the requested physician to request an appointment for the patient. In some embodiments in which the patient has not requested a specific physician, but rather has made a general request for an appointment, the scheduling server can send the request to a plurality of physicians, for example, those that satisfy certain criteria, such as preferences defined by the patient.

In some embodiments, a physician (or physicians) receiving the request, can view, for example, the name, the medical condition, the insurance information and the address of the requesting patient. To accept a request, a physician who has received an appointment request can accept or reject the requesting appointment. In some embodiments, a request for an in-office visit is automatically accepted (if the appointment slot is still available) while a request for a visiting appointment (e.g., a visit at the patient's house) may be accepted or rejected. As discussed in more detail below, a physician (or other medical professional) can employ a physician client application (herein also referred to as a “physician client app”) to accept or reject an appointment. A notice regarding the acceptance or rejection of an appointment is then transmitted to the scheduling server, which can in turn inform the patient, and more particularly, the patient client app, of the acceptance or rejection of the requested appointment.

Various embodiments discussed below provide further understanding of various aspects of the present teachings. It should be understood that although in many embodiments, various features of the present invention are discussed in connection with scheduling medical appointments with physicians, the present invention can be employed for scheduling appointments with other medical professionals, such as dentists, nurse practitioners, speech therapists, physical therapists, etc.

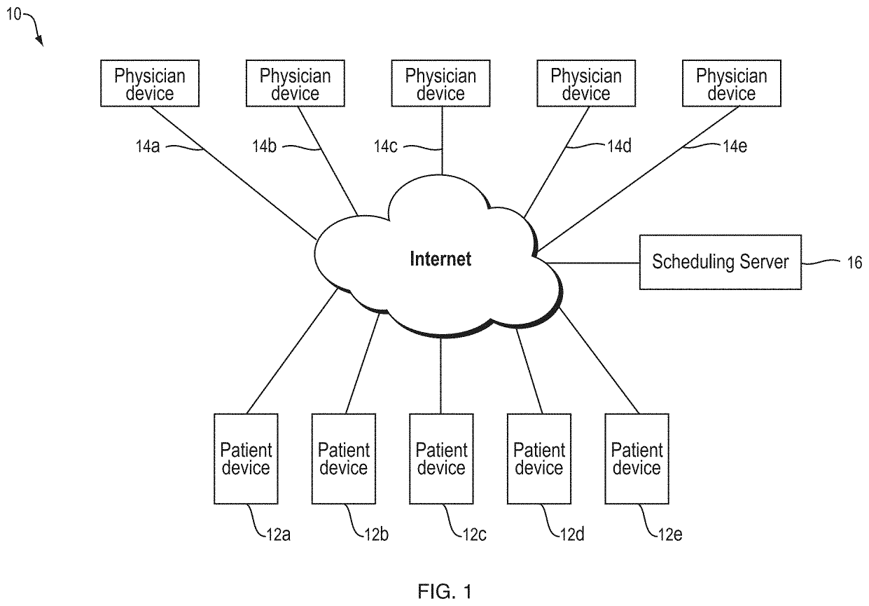

FIG. 1 schematically depicts a system 10 for scheduling medical appointments according to an embodiment of the present invention. In particular, the system 10 allows presenting available time slots to a plurality of patients for scheduling appointments with a plurality of medical professionals (e.g., physicians) and allows those patients to choose one or more of those time slots for scheduling appointments. In some cases, the available time slots are associated with canceled patients' appointments. In other words, in some cases, the time slots presented to patients are those that have become available due to cancellations. In other cases, the available time slots are those that have not yet been filled. As discussed in more detail below, a system according to the present teachings generates an electronic marketplace for scheduling medical appointments, which allows patients to schedule medical appointments on demand and further allows medical professionals to fill available appointment time slots much more efficiently than possible using conventional systems and methods. Although in many embodiments discussed below, the available time slots are discussed in the context of cancellations, it should be understood that the present teachings are applicable to appointment time slots that are available due to any reason.

For example, as discussed in more detail below, the system 10 can allow a physician to present available appointment time slots within the next 24 or 48 hours to a plurality of patients and allow those patients to choose one or more of the time slots to schedule appointments. In this manner, the physician can fill the available time slot(s), e.g., those associated with canceled appointments, and the patients are presented with the opportunity to schedule appointments on demand.

The system 10 includes a plurality of devices 12a, 12b, 12c, 12d, 12e (herein collectively referred to as “patient devices 12”) each of which is associated with a patient. As discussed in more detail below, a patient can employ a patient device 12 to schedule medical appointments on demand. Although for ease of illustration, only a few patient devices are shown, it should be understood that a system according to the present teachings is scalable for use by any number of patients. The patient device 12 can be any suitable device, e.g., a mobile device, such as a portable phone, a computer, e.g., a tablet, an iPad, a desktop, or any other device capable of communicating with a scheduling server, via a messaging network (e.g., the Internet), according to the present teachings.

The system 10 further includes a plurality of devices 14a, 14b, 14c, 14d, 14e associated with participating medical professionals (e.g., physicians, dentists, chiropractors, podiatrists, optometrists, etc.), which are herein referred to as “physician devices 14”. Each physician device can be associated with a physician (or any other medical professional, such as a dentist), and/or a physician's or other medical professional's practice, and/or an organization, such as a hospital, with which the physician or the medical professional is associated.

As discussed in more detail below, the physician devices can communicate with the patient devices, e.g., via a messaging network, to allow the scheduling of appointments. Again, although for the ease of illustration, only a few physician devices are shown, a system according to the present teachings can accommodate any number of physician devices.

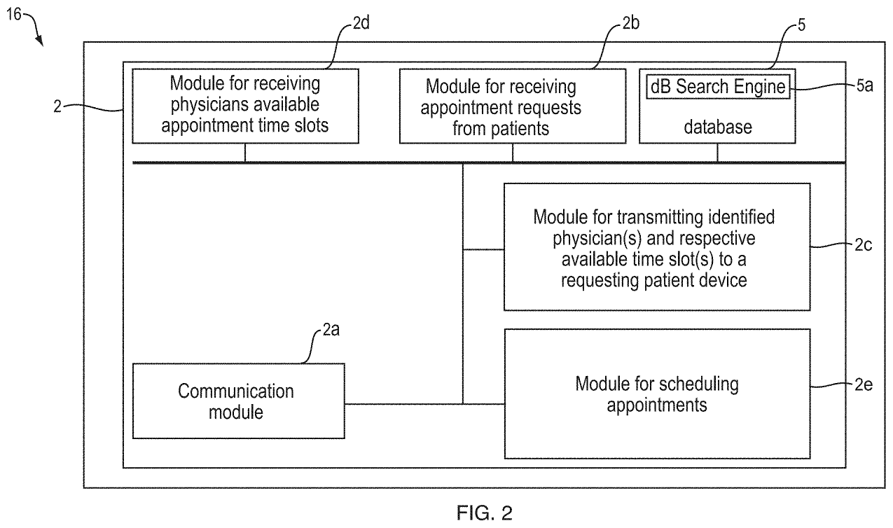

More particularly, as shown in FIG. 2, the system 10 includes a scheduling server 16 that can communicate with the physician devices and the patient devices to facilitate the scheduling of medical appointments, in a manner discussed in more detail below.

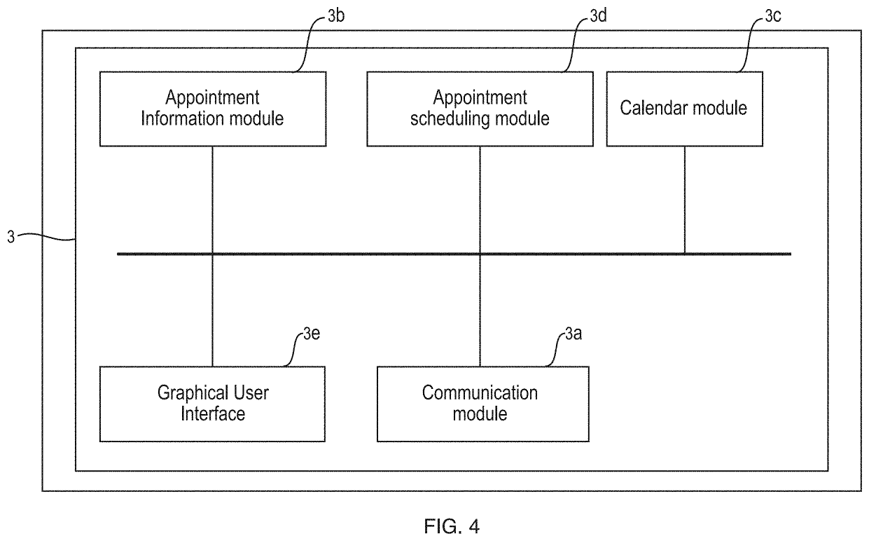

With reference to FIGS. 2, 3 and 4, each patient device 12 can include a patient client program (patient client application) 1 executing on the patient device that can communicate with a server program (server application) 2 executing on the scheduling sever 16 according to the present teachings. Similarly, with reference to FIG. 4, each physician device can include a physician client program (physician client application) 3 that can communicate with the server program 2. In this embodiment, each patient device 12, physician device 14, and the scheduling server 16, include communication modules 1a, 2a, and 3a that allow these devices to communicate with the scheduling server using systems and protocols known in the art.

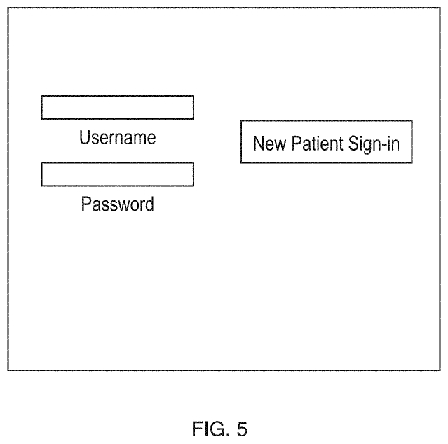

In this embodiment, the patient client program 1 allows a patient to register with the system and can further allow the patient to build a patient profile. By way of example, the patient client program 1 can present a graphical user interface (GUI) to the patient, which the patient can employ to interact with the client program 1, e.g., to register and compile his/her profile. By way of example, once the client program is launched, it can present an interface such as that depicted in FIG. 5 to the patient. The interface allows a patient, who has already registered with the system, to sign in using previously selected username and password. Further, the interface allows a new patient to begin the process of registration and building his/her patient profile.

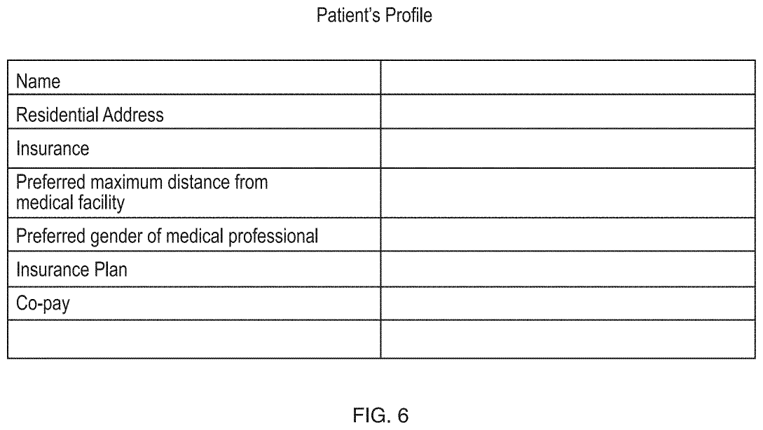

Once a new patient chooses the registration option, the patient client program can present a questionnaire to the patient that allows the patient to input patient data, e.g., biographical data. As shown schematically in FIG. 6, such patient data can include, for example, patient's name and address. Further, in this embodiment, the patient can input his/her preferences regarding the maximum distance of a medical professional from his/her address. As noted above and discussed in more detail below, in some embodiments, the present system can utilize such a geographical preference to filter the medical professionals of interest to a patient who have available appointment time slots within a time interval desired by the patient. In addition, in some embodiments, during the process of registration, the patient client program can present a liability waiver form to the patient for execution by the patient. Further, in this embodiment, the patient is requested to provide information regarding his/her insurance plan (including any co-pay) while building his/her profile.

Referring to FIG. 2, once a patient profile is completed, the patient client program can transmit the patient profile data to the scheduling server 16 and the scheduling server program 3 executing on the scheduling server 16 can store such patient profile data in a database 5 for use in future processing of the patient's requests. Thus, the database 5 can contain profiles of patients who have registered with the system.

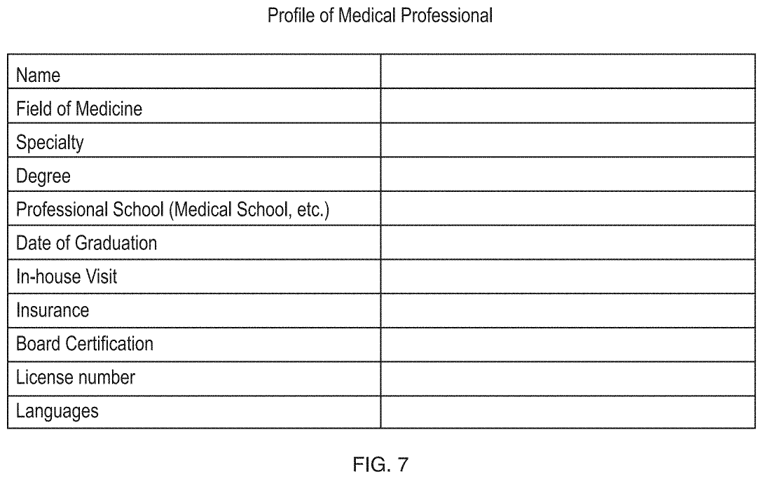

Referring again to FIG. 4, the physician client program 3 executing on a physician device 14 allows a physician, or other medical professional, to register with the system and provide information for preparing the physician's profile. For example, the physician client program 3 can present a form, such as that shown in FIG. 7, to request information regarding the participating physician (or other medical professional). For example, the physician can enter her/his name, the date of graduation from medical school, the physician's specialty, information about board certification, the physician's license number, the insurance plans accepted by the physician, whether or not the physician is willing to entertain in-house patient visits, languages spoken, etc. The physician client program 3 can transmit the information provided by the physician to the scheduling server 16 to be stored by the server scheduling program 2 in the database 5. Thus, in addition to patients' profiles, the physicians' profiles can also be stored in the database for future use.

Further, each physician client application (program) 3 can communicate with the scheduling server 16, and more specifically with the scheduling server program 2, to provide the scheduling server with information regarding available appointment time slots for that physician. As noted above, in some cases, these appointment time slots can correspond to appointments that have been canceled by one or more patients and in other cases they can correspond to appointment time slots that have not been yet filled.

As shown schematically in FIG. 4, in this embodiment, each physician client application 3 includes an appointment information module 3b, which can be in communication with a calendar module 3c residing on that device, or another device in communication with the physician device, to receive information regarding available appointment time slots for that physician (or other medical professional), such as canceled appointments.

The appointment information module 3b can then communicate information regarding the available appointment time slots to the scheduling server 16, and more specifically to the scheduling server program 2, which can in turn store the information in the database 5. As discussed below, the scheduling server 16 can use this information in response to a patient's request for scheduling an appointment to present available options to that patient.

With continued reference to FIG. 4, for example, in some embodiments, once a patient appointment is canceled, the calendar module 3c can send a notification to the appointment information module 3b regarding that cancellation. The appointment information module 3b can in turn use a communication module 3a of the physician's device to communicate the cancellation information to the scheduling server 16.

Alternatively, a user, e.g., a physician's assistant, can manually enter the information regarding the available appointment time slots, e.g., canceled appointment(s), into the appointment information module 3b, which can in turn transmit the information to the scheduling server 16. For example, such a user can employ a user interface unit 3d of the physician's device 14 to enter information regarding the canceled appointment(s), e.g., date and time of the canceled appointment(s), into the appointment information module 3b.

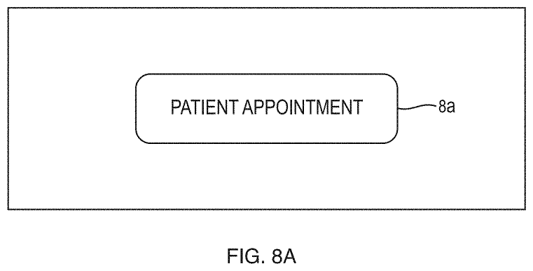

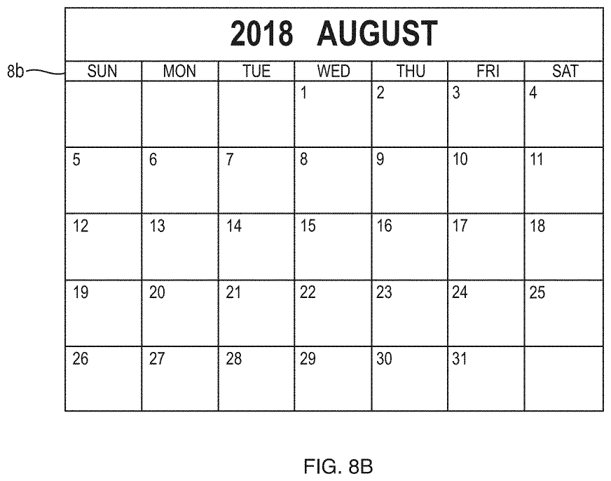

For example, as shown in FIG. 8A, the user interface unit 3d can present an icon 8a associated with the appointment information module 3b to the user. The user can select the icon 8 to launch an instantiation of an executable program residing on the appointment module 3b. This can in turn result in presentation of an appointment calendar 8b to the user. The user can select a date/time slot to update the calendar, e.g., to indicate a canceled appointment thereby generating an available appointment time slot. The appointment information module 3b can in turn store the updated date/time slots for available appointments and transmit that information to the scheduling server 16.

Although in some embodiments, the functionality of a system according to the present teachings is described in the context of canceled appointments, more generally, a system according to the present teachings can be employed to match a patient seeking a medical appointment with a physician having an appointment time slot meeting the patient's criteria, whether that appointment time slot is available due to a cancellation or not, e.g., the appointment slot may not have been filled in the first instance.

A patient can employ a patient device 12 to submit a scheduling request to the scheduling server program 2 executing on the scheduling server 16. In response to the received scheduling request, the scheduling server, and more specifically, the scheduling server program 2, can consult the database 5 to identify one or more physicians (or other medical professionals) that have available appointments that satisfy the scheduling request submitted by the patient.

Referring to FIG. 2, in this embodiment, the scheduling server 16 includes module 2b for receiving appointment requests from patients, which is in communication with a database search engine 5a associated with the database 5 to send a patient's request including the criteria identified by the patient for the physician (or other medical professional) to the database search engine 5a. The database search engine can then identify one or more physicians that satisfy the search criteria, e.g., medical specialty, distance from a patient location, etc. The database search engine 5a can provide the search results to a module 2c, which transmits the search results to a requesting patient's device for presentation to the patient.

More specifically, in this embodiment, the scheduling server 16 is in communication with a plurality of physicians devices 14a, 14b, 14c, 14d, 14e (herein referred to collectively as “physician devices”), each of which is associated with a physician and/or a physician's practice, and/or an organization, such as a hospital, with which the physician is associated. By way of example, in this embodiment, the scheduling server 16 can communicate with the physician devices via a messaging network, such as the Internet, though other modes of communication can also be employed.

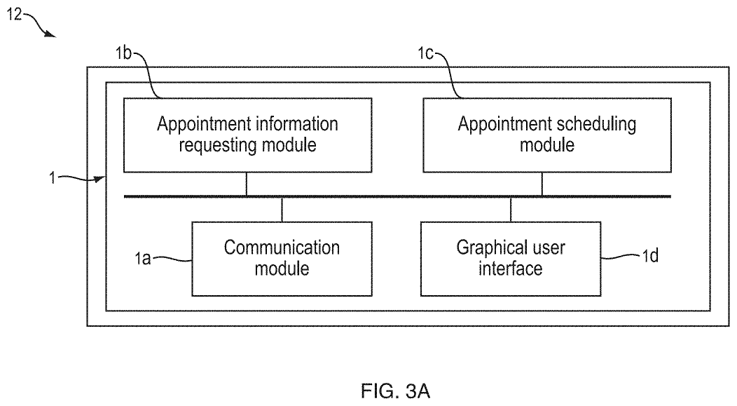

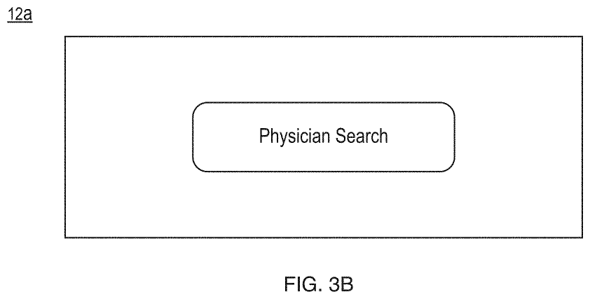

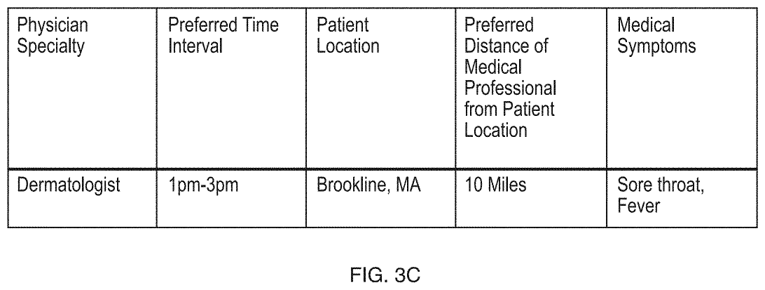

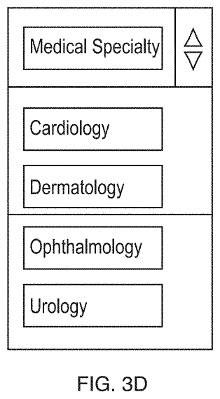

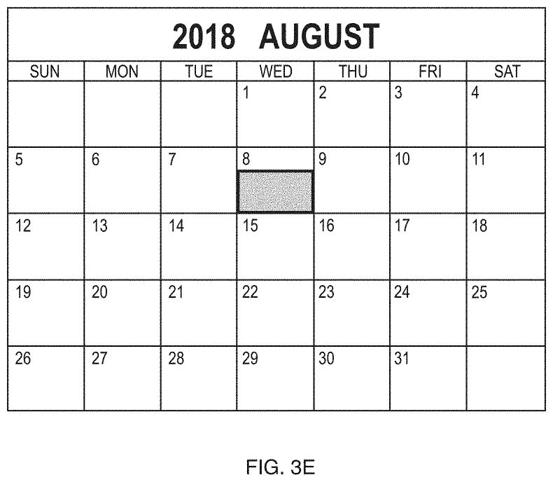

By way of example, with reference to FIG. 3A, the patient associated with the patient device 12a can use the patient client program 1 residing on the device 12a to request scheduling of an appointment with a dermatologist within the next 24 hours.

By way of example, as shown in FIG. 3B, the patient device 12a can include a display that presents an icon labeled “Physician Search,” associated with the patient client program 1. Upon selecting the icon “Physician Search,” e.g., by clicking on the icon, the patient client program 1 can present to the patient a graphical user interface (GUI) shown in FIG. 3C having a plurality of fields for soliciting information from the patient. For example, in this embodiment, the GUI includes fillable fields soliciting information from the patient regarding the medical specialty of a physician needed by the patient, and the date at which an appointment is needed. In addition to the date, in some embodiments, the GUI can allow a patient to provide a preferable time interval during which an appointment is desired.

In some embodiments, the GUI can also allow the patient to identify the maximum distance of the physician's office from the patient. For example and only for illustrative purposes, we assume that the date at which the patient lodges the request is Aug. 23, 2018 and the patient is seeking to schedule an appointment with a dermatologist on Aug. 24, 2018. It should be understood that similar features can be used to search for other medical professionals, such as, dentists, physical therapists, etc. In some embodiments, the GUI can present a pull-down menu to the patient, which the patient can employ to select the type of medical professional, e.g., a physician or a dentist, with whom the patient would like to schedule an appointment. Further, in some embodiments, for each medical profession, the patient can be presented with sub-specialties from which the patient can make a selection.

As shown in FIG. 3D, in some embodiments, the GUI presents a drop-down menu indicating various medical specialties from which the patient can choose. In this embodiment, upon selection of a medical specialty, e.g., dermatology in this case, the GUI presents a graphical element in the form of a calendar to the patient, as shown in FIG. 3E. The patient can use the calendar to select a date at which the appointment is needed. In some embodiments, the patient can further identify a preferable time interval, e.g., from 1 pm to 5 pm, for scheduling the appointment.

Further, in some embodiments, the patient may be provided with the option, or alternatively be required, to provide his/her symptoms while requesting scheduling an appointment with a medical professional. For example, with reference to FIG. 3D, in this example, the patient has indicated that she has sore throat and fever.

With reference to FIG. 2, the patient client program 1 can transmit the patient's request to module 2b of the scheduling server 16 via the appointment information requesting module 1b and the communication module 1a of the patient client application 1. Upon receipt of the patient's request, the module 2b of the scheduling server 16 can communicate with the database search engine 5a to provide the patient's request to the database engine. As noted above, in this example, the requesting patient is interested in scheduling an appointment with a dermatologist having appointment time slots on Aug. 24, 2018, and preferably between 1 pm and 3 pm. By way of illustration, in this example, the database search engine 5a can identify two dermatologists, namely, Dr. Adam Smith, and Dr. Diane Jones, who have available appointment time slots at 10 am and 3 pm on Aug. 24, 2018, respectively. The database engine 5a sends this information to the module 2c of the scheduling server application 2, which in turn communicates this information via the communication module 2a of the scheduling server application 2 to the patient device of the requesting patient.

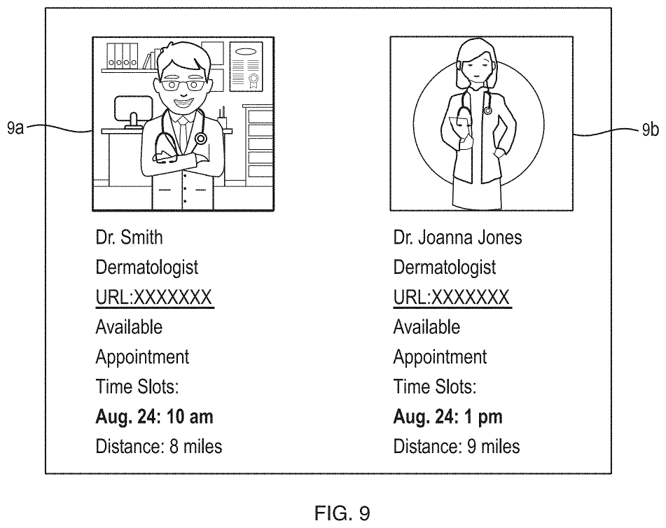

The patient client application executing on the patient device can in turn effect the presentation of this information via a display of the patient device to the requesting patient. For example, with reference to FIG. 9, in this embodiment, two icons 9a and 9a, each representing one of the available dermatologists, is presented to the patient. The date and time slot at which each of the dermatologists is available is also presented to the patient above, or below, or over the icon. In addition, for each physician, the distance from the patient's location is indicated. In other embodiments, the information regarding the available dermatologists can be presented to the patient in a tabular format. Other ways of presenting such information to the patient can also be employed.

In this embodiment, under each icon, a link to the physician's website as well as the available appointment time slots for that physician are provided.

The patient can select one of the two dermatologists and request scheduling an appointment, e.g., by clicking the available appointment time slot. The patient client program can then transmit the patient's request for scheduling an appointment to the scheduling server application 2 executing on the scheduling server 16. A module 2e for scheduling appointments executing on the scheduling server can receive the request and can determine, via communication with the database 5, whether the appointment slot is still available. If the selected time slot is available, the program module 2e can communicate with the appointment scheduling module 3d executing on the dermatologist's physician client application (See, FIG. 4) to schedule an appointment at the selected time slot. The appointment scheduling module 3d executing on the physician device can communicate with the calendar module to ensure that the scheduled appointment is reflected on the physician's calendar. The scheduling module 3d can also inform the scheduling server 16, and more particularly, the module 2e for scheduling appointments executing on the scheduling server that the appointment has been scheduled. The module 2e can in turn communicate this information to the database 5 to update the information stored in database 5 regarding available appointment time slots.

The module 2e can also send a confirmation notice to the requesting patient's device. For example, the module 2e can send the confirmation notice to the appointment scheduling module 1c executing on the patient's device, which can employ the graphical user interface of the requesting patient's device to present the confirmation notice to the patient.

In some embodiments, the system allows a patient seeking an appointment to filter the physicians who have available appointment time slots based on the maximum distance from a selected location, e.g., a patient's residence, to the medical facility at which the physicians practice. For example, as discussed above and as shown in FIG. 3C, the patient client program can provide an option to the patient to input an address, e.g., the patient's location, and a maximum distance from that address. The client program can transmit this information to the scheduling server 16 and the scheduling server 16 can use this information to filter the physicians who have available appointment time slots that would fit the patient's criteria so as to present to the patient only those physicians who are located at a distance from the selected address that is at most equal to the selected distance.

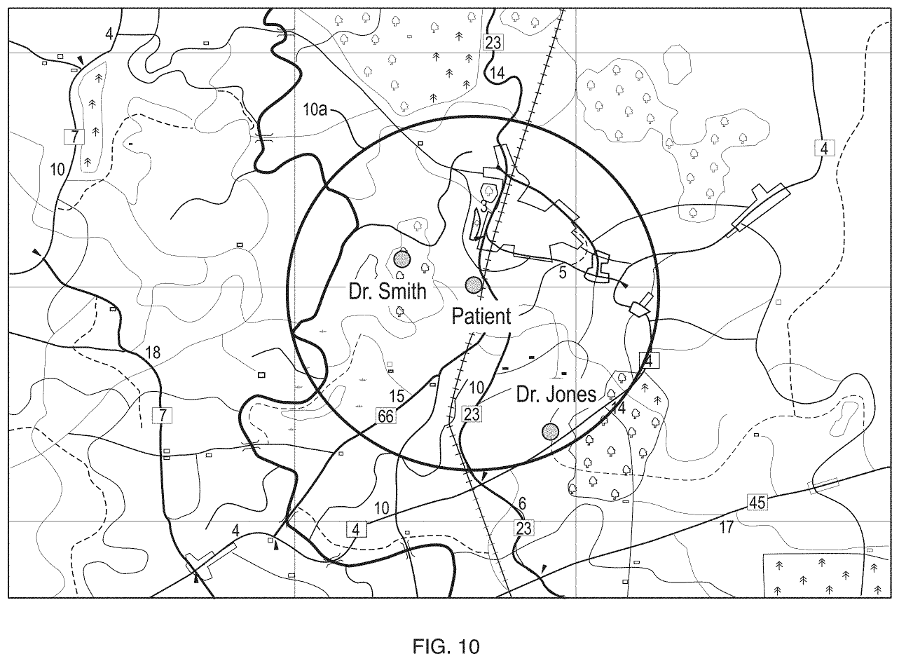

The scheduling server 16 can then transmit information regarding the physicians whose availability and distance fit the criteria selected by the patient to the patient client application. For example, FIG. 10 shows that in some embodiments, the client program shows the locations of the available physicians on a map within a circle 10a whose origin is the address selected by the patient, e.g., the patient's residential address, and whose radius is the maximum distance selected by the patient.

In some embodiments, the system can employ a default maximum address to filter the physicians who have available appointment time slots. For example, the default maximum address can be about 20 miles from a patient's residential address, which the patient had previously provided in registering with the system.

In some other embodiments, the system can filter the medical professionals (e.g., physicians) presented to a patient seeking an appointment based on the patient's insurance plan and whether or not a physician is within the network of the patient's insurance plan. As discussed above, in many embodiments, a patient provides the system with information regarding the patient's insurance plan when the patient registers with the system, e.g., in a manner discussed above.

Upon receiving an appointment request from the patient, the server program filters the physicians who have available time slots fulfilling the patient's criteria based on whether those physicians accept the patient's insurance plan. For example, in some embodiments, the scheduling server 16 provides the requesting patient with information regarding only those physicians who accept the patient's insurance plan.

In some embodiments, if a patient's request for an appointment with a medical professional, e.g., a physician, within a selected time period cannot be fulfilled because no appointment time slots are available, the system can provide the patient with the option of requesting a notification when a time slot becomes available, e.g., a notification as soon as a time slot becomes available. By way of example, in some such embodiments, the client program may present a message to the patient such as that shown indicating that no appointment slots are available that would satisfy the criteria provided by the patient. Further, the patient client application can provide the patient with the option of requesting a notification when an appointment slot becomes available. If the patient requests such a notification, the patient client application can transmit the patient's request to the scheduling server and the scheduling server program can provide such a notification to the client program once an appointment slot becomes available.

In a related aspect, a system according to the present teachings can allow patients to request in-house visits. For example, upon registration with the system, a physician (or other medical professional) can indicate whether she/he is available for in-house visits. In some such embodiments, the patient client application can provide a patient with the option of requesting an in-house visit. For example, the patient client program can present a tab labeled “In-house Visit,” which a patient can select to request an in-house visit. Upon selecting the “In-house Visit” tab, the patient client application can provide, for example, a pull-down menu (such as that shown in FIG. 3D) from which the patient can select a medical specialty of interest to the patient. Similar to the previous embodiments, upon selecting a medical specialty, the patient client application can provide the patient with a graphical interface, which allows the patient to identify a range of dates and times during which the patient would desire to have an appointment. The patient client application can transmit this information to the scheduling server and the scheduling server can identify those physicians who are available for in-house visits and who have available time slots that meet the criteria identified by the patient. The server can in turn transmit a list of available physicians to the client program for presentation to the patient. The patient can in turn select a physician of interest and request an appointment with that physician.

In some embodiments, a patient scheduling system according to the present teachings can be configured to provide priority to certain patient population before presenting the available appointment time slots to a wider group of patients. By way of example, the system can initially post available time slots to a physician's or other medical professional's own patients, e.g., those on a waiting list for obtaining an appointment, and then after a given time period, e.g., a few hours, make those appointment slots available to the general population.

By way of example, a physician device can transmit, e.g., on a periodic basis (e.g., on daily basis), a list of patients to the scheduling server for whom priority scheduling is required. The scheduling server can associate the information regarding those patients with that physician and store that information in the database 5. In some such embodiments, the scheduling server will present the available appointment time slots only to the priority patients during a specified time period, e.g., a few hours. After the termination of the specified time period, the scheduling server can present the available time slots to other patients.

The physician device can update the list of priority patients and available time slots. For example, when a patient may call a physician's office directly to make an appointment, if an appointment is made, the physician device can update the available appointment time slots and send the updated information to the scheduling server. The scheduling server can in turn update the database 5 accordingly.

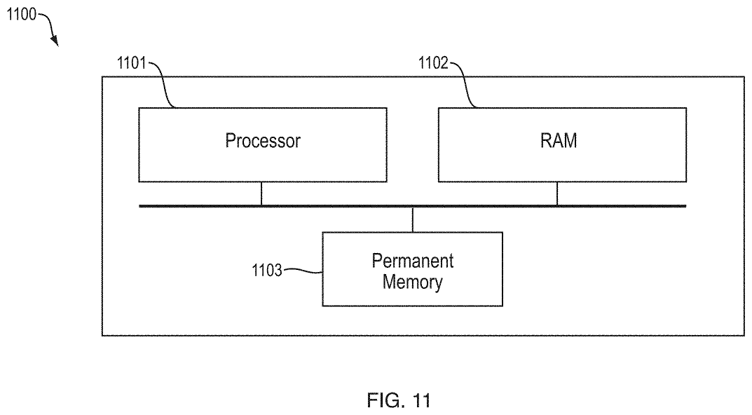

Any of the scheduling server as well as the physician and patient client applications can be implemented on a digital processor using hardware, firmware and/or software in a manner known in the art as informed by the present teachings. By way of example, FIG. 11 schematically depicts such an implementation 1100, which includes, among other elements known in the art, a digital processor 1101, a random access memory (RAM) module 1102, a permanent memory module 1103, and a communications bus 1104 that allows the digital processor to communicate with the memory modules to execute various instructions stored in the memory modules. By way of example, the instructions to implement the present teachings can be stored in the permanent memory module 1103. During execution, the processor can effect the transfer of these instructions to the RAM 1102 for run-time execution. The instructions can be implemented using any known programming language, such as, Python, Java, C++ or any other suitable programming language.

Those having ordinary skill in the art will appreciate that various changes can be made to the above embodiments without departing from the scope of the invention.

What is claimed is:

1. A computer-implemented system for scheduling medical appointments, comprising,
a scheduling server, implemented on a digital processor, and configured to communicate with a plurality of patient devices associated with a plurality of patients and a plurality of physician devices associated with a plurality of physicians,
said scheduling server program comprising:
a first module for receiving requests from said patient devices for information regarding at least one available physician having at least one available appointment time slot,
a second module for receiving available patient appointment time slots from said plurality of physician devices, said second module storing information regarding said available appointment time slots,
a third module in communication with said first module for searching said database in response to a request from at least one of said patient devices to identify one or more physicians and one or more available appointment time slots associated with said one or more physicians that satisfy said request, and
a fourth module in communication with said third module for receiving information regarding said identified physicians and the respective available time slots from said third module and for transmitting said information to said at least one requesting patient device.

2. The system of claim 1, wherein each of said physician devices comprises
an appointment information module for communicating available appointment time slots to said scheduling server, and
an appointment scheduling module for scheduling an appointment at a time slot selected by a patient.

3. The system of claim 1, wherein each of said patient devices comprises:
a first module for communicating a patient's request for identifying at least one physician having at least one available appointment time slot within a desired temporal period to said scheduling server, said first module further configured for receiving information regarding available physicians and associated available appointment time slots from said scheduling server,
a graphical user interface (GUI) in communication with said first module for presenting said information regarding available physicians and associated available time slots to the patient.

4. The system of claim 2, wherein each of said patient devices comprises a second module in communication with said GUI to receive a patient's selection of an available physician and a respective available time slot and to communicate said selection to said scheduling server.

5. The system of claim 3, wherein said scheduling server further comprises a fifth module in communication with said second module of each of said patient devices for receiving a patient's selection of an available physician and a respective available appointment time slot and to send an appointment scheduling request for said time slot for said requesting patient to the physician device associated with said available physician.

6. The system of claim 3, wherein said GUI employs a graphical element for presenting said information regarding available physicians to the patient.

7. The system of claim 6, wherein said graphical element presents a boundary line circumscribing an area within a maximum distance from a patient's address and further presents one or more icons each indicative of a physician having an available appointment time slot within said area.

8. The system of claim 7, wherein each icon is positioned at a distance from the patient's address proportional to an actual distance of the presented physician from the patient's address.

9. The system of claim 8, wherein said icon is configured such that selection thereof results in presenting information regarding the physician associated with said icon to the patient.

10. The system of claim 6, wherein said GUI superimposes a map on said circumscribed area and marks said patient's address and one or more addresses of said one or more physicians on said map.

11. A computer-implemented method of scheduling medical appointments, comprising:
using at least one digital data processor to perform the following steps:
receiving at least one request from at least one patient device for information regarding at least one available physician having at least one available appointment time slot,
receiving from one or more physician devices information regarding one or more available appointment time slots associated with said one or more physicians,
comparing said available appointment time slots with said patient-requested time slot to determine if any of the available appointment time slots satisfies the patient's request, and
presenting at least one available appointment time slot satisfying the patient's request, if any, to the patient.

12. The computer-implemented method of claim 11, wherein said received one or more available time slots correspond to appointment time slots made available via patient cancellation.

13. A computer-implemented system for scheduling medical appointments, comprising:
a scheduling system comprising a digital data processor for receiving at least one request from a patient for a medical appointment with a medical professional at a selected date and time interval,
an information-receiving system comprising a digital data processor and configured for receiving information regarding one or more physicians having available appointment time slots at said selected date and time interval and communicating said information to the scheduling system,
wherein said scheduling system presents said available appointment time slots to said patient.

14. The system of claim 13, wherein the scheduling system is further configured to receive from a patient a designation of a patient's address and a maximum distance and to filter the one or more physicians having available appointment time slots at said selected date and time interval to only those located within the maximum distance of the patient's address.
